(19) United States
(12) Patent Application Publication
(10) Pub. No.: US 2026/0325492 A1
(43) Pub. Date: Oct. 22, 2026

(54) EXTENDED-RELEASE FORMULATION OF NITROFURANTOIN AND METHODS OF HYBRID COATING OF NITROFURANTOIN PELLETS

(71) Applicants: Ramya SRI Sura, Monmouth Junction, NJ (US); Sura Pharma Labs, Hyderabad (IN)

(72) Inventor: RAMYA SRI SURA, Monmouth Junction, NJ (US)

(21) Appl. No.: 19/307,061

(22) Filed: Aug. 22, 2025

(51) Int. Cl. A61K 31/4178; A61K 9/48; A61K 9/50

(52) U.S. Cl. CPC A61K 31/4178; A61K 9/4866; A61K 9/501; A61K 9/5015; A61K 9/5026; A61K 9/5047; A61K 9/5078; A61K 9/5089

(57) ABSTRACT

The present disclosure discloses an extended-release formulation of nitrofurantoin and methods of hybrid coating of nitrofurantoin pellets. The pharmaceutical formulation includes pellets with nitrofurantoin as an active ingredient and an extended-release coating made of a mixture of ethyl cellulose and methacrylic acid-ethyl acrylate copolymer. Ethyl cellulose is used as a release-controlling polymer, and methacrylic acid-ethyl acrylate copolymer is used as delayed release polymer or enteric coating polymer. The release-controlling polymer is 60%-95% of the polymer mixture, while the delayed release polymer is 05%-40% of the polymer mixture. The method involves preparing drug-layered pellets with nitrofurantoin as an active ingredient, applying a seal coating to obtain sealed-coated pellets, and applying a hybrid extended-release coating using a mixture of ethyl cellulose and methacrylic acid-ethyl acrylate copolymer.

### FIELD OF THE PRESENT INVENTION

The present disclosure relates to the field of pharmaceutical sciences, particularly relates to an extended-release formulation of nitrofurantoin and methods of hybrid coating, i.e., a mixture of delayed release and extended-release polymers, of nitrofurantoin pellets.

### BACKGROUND OF THE DISCLOSURE

Urinary tract infections (UTIs) are among the most common bacterial infections worldwide, with an estimated 150 million cases reported annually. The growing prevalence of drug-resistant strains, including carbapenem-resistant pathogens, has led to an increased reliance on nitrofurantoin as a first-line treatment. Nitrofurantoin is effective against a range of uropathogens, including Escherichia coli, enterococci, Staphylococcus aureus, and certain strains of Klebsiella and Enterobacter.

Despite their effectiveness, current nitrofurantoin formulations present significant limitations that hinder optimal patient outcomes. Existing immediate-release capsules and suspensions require frequent dosing, typically four times daily, which can be burdensome for patients and leads to poor adherence. Other alternatives, such as microcrystal slow-release formulation, designed for twice-daily dosing, rely on slower solubility rather than true sustained drug delivery.

Frequent dosing reduces patient compliance and also increases the risk of treatment failure and recurrence, especially for populations requiring long-term use. Immediate-release nitrofurantoin causes dose-dependent gastrointestinal side effects, such as nausea, vomiting, anorexia, abdominal pain, and diarrhea. Adverse effects further compromise treatment adherence and patient comfort.

Efforts to develop sustained- or controlled-release formulations of nitrofurantoin have been unsuccessful due to a lack of precise control over release onset.

A non-patent literature titled “Extended-Release Tablets of Nitrofurantoin: An Old Antibiotic Carried in a New Formulation, “Published on 19 Nov. 2021, discloses tablets prepared using nitrofurantoin and crosslinked poly(acrylic acid) and hydroxypropyl methylcellulose (HPMC) as hydrophilic polymer matrices.

Another nonpatent literature titled “Sustained release of nitrofurantoin from inert wax matrixes” published on 8 Nov. 2002 discloses sustained release nitrofurantoin granules containing stearic acid and glyceryl monostearate (matrix) that are prepared using fusion, solvent evaporation, or melt granulation techniques. The effect of aerosil, avicel, dibasic calcium phosphate dihydrate, and sodium chloride as channeling agents on the in vitro release of nitrofurantoin is investigated. The results obtained revealed that with the granules prepared by the fusion method, a sustained release pattern can be achieved, contrary to those prepared by the other techniques. Moreover, all the selected channeling agents increase the extent of the drug release, but with different rates depending upon their concentration and nature. The release of the drug is found to follow the Higuchi model with granules containing 0 and 5% channeling agents, while it follows first-order kinetics with those containing 10% channeling agents. In vivo studies in humans revealed that the urinary excretion of nitrofurantoin from the test granules occurs over a sustained period from 2-8 hours after oral administration.

However, the above-mentioned arts do not disclose any composition or method to develop an extended-release formulation of nitrofurantoin to provide a single dose to cover the 24-hour window.

### BRIEF SUMMARY OF THE DISCLOSURE

One or more embodiments of the present disclosure relate to an extended-release formulation of nitrofurantoin and methods for preparing hybrid-coated nitrofurantoin pellets.

An embodiment of the present disclosure relates to a pharmaceutical composition. The pharmaceutical composition includes drug-layered pellets containing nitrofurantoin as an active pharmaceutical ingredient (API) and an extended-release hybrid coating of the pellets using a mixture of ethyl cellulose and methacrylic acid-ethyl acrylate copolymer. The ethyl cellulose acts as a drug release-controlling polymer, and methacrylic acid-ethyl acrylate copolymer (1:1), also known as Eudragit L100-55, acts as a delayed or enteric release polymer. The release-controlling polymer includes 60%-95% of the mixture, and the delayed release polymer comprises 05-40% of the mixture. The hybrid extended-release coating of pellets helps in following a sustained-release profile over 24 hours. In an embodiment, the ethyl cellulose polymer comprises 80% of the mixture, and the methacrylic acid-ethyl acrylate copolymer comprises 20% of the mixture.

In an embodiment, the extended-release coating includes dibutyl sebacate acting as a plasticizer, micro talc USP acting as an anti-tacking agent, and ethanol acting as a solvent. Further, the extended-release coating is applied to achieve a weight gain of up to 20% w/w.

An embodiment of the present disclosure discloses a method for preparing extended-release nitrofurantoin pellets. The method includes the steps of preparing drug-layered pellets comprising nitrofurantoin as an active ingredient, applying a Hypromellose USP, also called Methocel E5 Premium LV, as seal coating polymer over the drug-layered pellets, and applying extended-release coating comprising a mixture of ethyl cellulose and methacrylic acid-ethyl acrylate copolymer on the seal-coated pellets.

The drug-layered pellets are prepared by providing microcrystalline cellulose (MCC) spheres having a D90 particle size of 350-500 μm as an inert core substrate. A drug dispersion comprising nitrofurantoin monohydrate and the methocel E5 premium LV (binder) in purified water. Drug dispersion is layered on the MCC spheres using a Freund Vector VFC-LAB 1fluid bed processor, equipped with a bottom spray Wurster insert, to produce the drug-layered pellets.

In an embodiment, the method includes the steps of applying a seal coating over the drug-layered pellets to obtain a sealed-coated pellet. The seal coating is applied on drug-layered pellets by preparing a seal coating solution by dissolving Methocel E5 Premium LV in purified water. Micro Talc USP is dispersed in the hypromellose solution to form a seal coating dispersion.

In an embodiment, the hypromellose is an inert, water-soluble polymer also known as hydroxypropyl methylcellulose (HPMC), functioning primarily as a binder and film-forming agent. The hypromellose used is provided under the trade name Methocel™, specifically the E5 Premium LV grade. The “E5” designation corresponds to a low-viscosity grade of hypromellose, exhibiting a viscosity of approximately 5 mPa s in a 2% aqueous solution at 20 °C. The “Premium” label indicates pharmaceutical-grade quality with high purity, and “LV” denotes low viscosity, making it particularly suitable for use in aqueous film coating systems and as a granulation binder.

In an embodiment, micro talc USP is an inert anti-tacking agent. The seal coating dispersion is sprayed onto drug-layered pellets using a Freund Vector VFC-LAB 1fluid bed processor equipped with a Wurster coating assembly. The said seal coating is applied to achieve a target coating level of approximately 10% w/w.

In an embodiment, the method includes the steps of applying a hybrid extended-release coating over the seal-coated pellets using a mixture of ethyl cellulose and methacrylic acid-ethyl acrylate copolymer. The ethyl cellulose acts as a drug release-controlling polymer, and the methacrylic acid-ethyl acrylate copolymer acts as delayed or enteric release polymer. The release-controlling polymer comprises 60%-95% of the mixture, and the delayed release polymer comprises 05%-40% of the mixture. The extended-release coating is applied by preparing a hybrid coating composition by dissolving ethyl cellulose and methacrylic acid-ethyl acrylate copolymer in ethanol. Dibutyl Sebacate is added to the mixture, acting as a plasticizer. Talc is used as an anti-tacking agent. Optionally, additional water-soluble polymers can be used in the coating system to modulate the overall drug release profile. In an embodiment, the hybrid extended-release coating is applied by spraying the coating composition onto the seal-coated pellets using a Freund Vector VFC-LAB 1fluid bed processor equipped with a Wurster coating assembly. Continuing the coating process until a weight gain of up to 20% w/w is achieved.

In an embodiment, the ethylcellulose polymer used is supplied under the trade name Ethocel™, with commonly used grades including Ethocel Standard 10 Premium. Ethocel is a water-insoluble, organosoluble cellulose ether, derived by the ethylation of cellulose. The “10” grade refers to viscosity of approximately 10 mPa·s measured in a 5% solution in an 80:20 toluene/ethanol mixture at 25 °C, indicating a medium molecular weight. The “Premium” designation confirms that the polymer meets pharmaceutical standards with high purity and low residual solvents, making it suitable for use in sustained-release coatings, matrix tablets, and microencapsulation. Ethocel functions as a hydrophobic polymer, providing moisture protection and controlling the release of active ingredients in both aqueous and organic solvent-based systems.

In an embodiment, the methacrylic acid-ethyl acrylate copolymer used is marketed under the trade name Eudragit®, with several variants tailored for controlled drug release. One common grade is Eudragit L100-55, a methacrylic acid-ethyl acrylate copolymer (1:1). This polymer is anionic and exhibits pH-dependent solubility, dissolving at pH 5.5 and above, making it ideal for enteric coating applications. The “L100-55” designation indicates that the polymer dissolves in the lower intestine, protecting it from gastric fluids. Eudragit L100-55 is used to ensure the delayed release of active ingredients, enhancing targeted delivery and gastrointestinal stability.

The features and advantages of the subject matter here will become more apparent in light of the following detailed description of selected embodiments, as illustrated in the accompanying FIGURES. As will be realized, the subject matter disclosed is capable of modifications in various respects, all without departing from the scope of the subject matter. Accordingly, the drawings and the description are to be regarded as illustrative.

### BRIEF DESCRIPTION OF THE DRAWINGS

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label, irrespective of the second reference label.

FIG. 1 is an exemplary illustration of an individual pellet of the multi-particulate system, in accordance with an embodiment of the present disclosure.

FIG. 2 is a flowchart of producing nitrofurantoin extended-release pellets, in accordance with an embodiment of the present disclosure.

FIG. 3 illustrates a process for creating drug-layered pellets, in accordance with an embodiment of the present disclosure.

FIG. 4 illustrates a process for creating seal-coated pellets, in accordance with an embodiment of the present disclosure.

FIG. 5 illustrates a process for the extended-release coating, in accordance with an embodiment of the present disclosure.

Other features of embodiments of the present disclosure will be apparent from the accompanying drawings and detailed description that follows.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the broadest possible scope consistent with the principles and features disclosed herein.

Unless otherwise defined, all technical and scientific terms used herein have the same meaning as commonly understood by one of ordinary skill in the art to which this disclosure belongs. Methods and materials are described herein for use in the present disclosure; other suitable methods and materials known in the art can also be used. The materials, methods, and examples are illustrative only and are not intended to be limiting. All publications, patent applications, patents, sequences, database entries, and other references mentioned herein are incorporated by reference in their entirety. In case of conflict, the present specification, including definitions, will control.

### TERMINOLOGY

Brief definitions of terms used throughout this application are given below.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,”, “in an alternative embodiment” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

Exemplary embodiments will now be described more fully hereinafter concerning the accompanying drawings, in which exemplary embodiments are shown. This disclosure may, however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

As used herein, the term “antibiotic” refers to agents, compounds, or drugs that are used to treat or prevent infections caused by bacteria and other susceptible microorganisms. Antibiotics function through a variety of mechanisms, including inhibition of bacterial cell wall synthesis, interference with protein synthesis, disruption of nucleic acid replication, or inhibition of essential metabolic pathways. The term “antibiotic” as used herein includes both bactericidal agents (which kill bacteria) and bacteriostatic agents (which inhibit bacterial growth), and encompasses naturally derived, semisynthetic, and synthetic compounds. Exemplary antibiotic agents, compounds, or drugs include, but are not limited to, the following substances: nitrofurans such as nitrofurantoin and furazolidone; penicillins including penicillin G, penicillin V, amoxicillin, ampicillin, and dicloxacillin; cephalosporins including cephalexin, cefuroxime, cefaclor, ceftriaxone, cefepime, and ceftaroline; carbapenems such as imipenem, meropenem, doripenem, and ertapenem; monobactams such as aztreonam; aminoglycosides including gentamicin, tobramycin, amikacin, neomycin, and streptomycin; tetracyclines including tetracycline, doxycycline, and minocycline; macrolides including erythromycin, azithromycin, clarithromycin, and fidaxomicin; fluoroquinolones including ciprofloxacin, levofloxacin, moxifloxacin, and norfloxacin; sulfonamides such as sulfamethoxazole, sulfadiazine, and sulfisoxazole, often used in combination with trimethoprim; glycopeptides such as vancomycin and teicoplanin; lincosamides including clindamycin and lincomycin; oxazolidinones such as linezolid and tedizolid; rifamycins including rifampin and rifabutin; polymyxins such as polymyxin B and colistin; nitroimidazoles including metronidazole and tinidazole; and other agents such as chloramphenicol, fosfomycin, bacitracin, and daptomycin. This definition also includes pharmacologically acceptable salts, hydrates, solvates, esters, prodrugs, and other derivatives of the foregoing compounds, where applicable.

One or more embodiments of the present disclosure relate to an oral pharmaceutical composition. The oral pharmaceutical composition includes nitrofurantoin formulated as a delayed-release or extended-release dosage form for the treatment or prevention of urinary tract infections. The extended-release formulation is also known as sustained-release (SR), sustained-action (SA), time-release (TR), controlled-release (CR), modified-release (MR), or continuous-release (CR). The extended-release is a mechanism used in medicine tablets or capsules to dissolve slowly and release the active ingredient over time. The extended-release tablets or capsules are often taken less frequently than immediate-release formulations of the same drug, and they keep steadier levels of the drug in the bloodstream, thus extending the duration of the drug action.

In an embodiment, a hybrid coating is done to achieve a desired drug release profile by incorporating a combination of functional polymers within a single coating layer. The hybrid coating system comprises a combination of water-insoluble polymer and enteric polymer, such as methacrylic acid-ethyl acrylate copolymer (1:1) (e.g., Eudragit®L100-55), and at least one controlled-release polymer such as ethyl cellulose.

In an embodiment, the enteric component of the hybrid coating remains intact under gastric conditions and dissolves at pH values above approximately 5.5, thereby preventing drug release in the stomach and enabling pH-dependent drug release in the upper intestinal tract. Upon dissolution of the enteric polymer in the intestinal environment, the controlled-release component modulates drug diffusion, thereby enabling a sustained or delayed release effect. The hybrid coatings are applied using conventional aqueous or organic coating processes, including fluidized bed coating or pan coating techniques. The coating composition further includes one or more plasticizers, pore-formers, or anti-tacking agents. The use of a hybrid coating system is particularly advantageous for oral dosage forms requiring site-specific delivery, chronotherapeutic control, or improved pharmacokinetic performance through modified or delayed drug release. In an embodiment, the enteric polymer is a pH-sensitive polymer that remains insoluble in stomach acid but dissolves at higher pH levels in the small intestine. Commonly used in pharmaceutical formulations to create enteric coatings to protect active pharmaceutical ingredients from degradation or prevent gastric irritation. These polymers, like methacrylic acid-methyl or ethyl acrylate copolymers, facilitate site-specific drug delivery, minimize gastric side effects, and target intestinal absorption. The enteric coating functions as a barrier layer, physically separating the core drug material from gastric fluids. When the coated pellets are ingested, the enteric polymer remains intact in the stomach, resisting disintegration and preventing premature drug release. Upon transition to the small intestine, the rise in pH triggers polymer ionization, leading to hydration, swelling, and dissolution of the coating film. This process creates pores or complete erosion of the coating, thereby allowing gastrointestinal fluids to penetrate the core and initiate drug release.

In an embodiment, plasticizers are added to polymer coatings to enhance flexibility, reduce brittleness, and improve film-forming properties. They function by lowering the glass transition temperature (Tg) of the polymer, thereby making the film more elastic and resistant to cracking. Common examples of plasticizers include triethyl citrate (TEC), diethyl phthalate (DEP), polyethylene glycol (PEG 400), and propylene glycol.

In an embodiment, pore-formers are incorporated into coating formulations to create microporous structures within the polymer matrix. The pores allow the controlled passage of gastrointestinal fluids into the dosage form or facilitate the release of the active pharmaceutical ingredient. Pore-formers can be either water-soluble additives or leachable agents. Examples include sodium chloride, lactose, hydroxypropyl cellulose (HPC), and polyvinylpyrrolidone (PVP).

In an embodiment, anti-tacking agents are used to prevent sticking or agglomeration of coated particles, tablets, or pellets during the coating process. They reduce the tackiness of the wet coating and promote better handling and processing. Common anti-tacking agents include talc, magnesium stearate, glycerol monostearate, and colloidal silicon dioxide. These additives help ensure smooth operation during scale-up and improve the uniformity and quality of the final coated product.

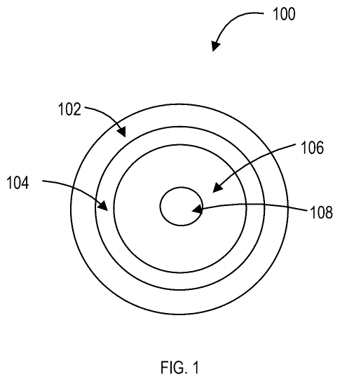

FIG. 1 is an exemplary illustration of an individual ER pellet 100 of the multi-particulate system in accordance with an embodiment of the present disclosure. The individual ER pellet 100 of the multi-particulate includes a drug-coated layer 106, a microcrystalline cellulose (also known as MCC sphere) 108, onto which the drug-coated layer is applied, a seal-coated layer 104, and an extended-release hybrid coated layer 102.

In an embodiment, the pellets have several benefits, including dosage uniformity and the option of taking medication in a sprinkle form with food. They are developed and modified through the pelletization technique, ensuring uniform drug release. Additionally, pellets contain a coating to mask the bitter taste and smell, making them more palatable. In an embodiment, pelletization is a process used to create pellet drugs, with various techniques being employed depending on demand and feasibility. The extrusion-spheronization technique is widely used by many pharmaceutical companies. The technique includes mixing dry active ingredients, creating a wet mass, shaping it into a cylindrical shape, and spheronizing it into a spherical shape. In an embodiment, another technique is hot-melt extrusion, which uses a liquid or semi-liquid form instead of dry powder, resulting in increased efficiency. In an embodiment, another technique is drug layering, which uses an inert core, and the drug is deposited on top of the inert core or pellet, and it's further coated using polymeric material. The pellets are filled into capsules and or compressed into tablets, with many pharma manufacturing companies focusing on pellet drugs due to their reliability and safety.

In an embodiment, the hybrid extended-release coating 102 releases the active ingredient at a predetermined rate to maintain therapeutically effective drug levels over an extended duration, such as up to about 24 hours following administration. The hybrid extended-release coating 102 provides sustained or controlled release of the active ingredient over a time ranging from about 0 hours to about 24 hours, including, but not limited to, about 12 hours, about 16 hours, about 18 hours, about 20 hours, or about 22 hours. In an embodiment, the active ingredient is released over approximately 24 hours to enable once-daily dosing. The active ingredient is released over any interval ranging from about 0 hours to about 24 hours, depending on the desired pharmacokinetic profile and therapeutic effect.

In an embodiment, the hybrid extended-release coating composition provides a sustained release of nitrofurantoin over a 24-hour period, which enables once-daily dosing. This prolonged release helps maintain therapeutic plasma and urine concentrations of the drug over the entire dosing interval, thereby improving patient compliance and minimizing peak-trough fluctuations that are typically associated with immediate-release or multiple-dose formulations. The 24-hour profile reduces the frequency of administration from four doses per day (in conventional nitrofurantoin regimens) to a single daily dose, which can be particularly beneficial for elderly patients or those requiring long-term therapy for recurrent urinary tract infections.

In an embodiment, the hybrid extended-release coating 102 includes an active core. The active core includes one or more inert particles. Each of the one or more inert particles is in the form of a bead, pellet, pill, granular particle, microcapsule, microsphere, microgranule, nano capsule, or nanosphere coated on its surfaces with drugs in the form of e.g., a drug-containing coating or film-forming composition using, for example, fluid bed techniques or other methodologies known to those of skill in the art. The inert particle can be of various sizes, so long as it is large enough to remain intact during the manufacturing process. In an embodiment, the active core may be prepared by granulating and milling and/or by extrusion and spheronization of a polymer composition containing the drug substance.

In an embodiment, the seal coating 104 of pellets is applied on top of the drug layered coating 106 to reinforce their surface and protect them from mechanical damage, such as breakage during subsequent processing steps like fluidization in fluid bed coaters. Seal coating 104 also serves as a protective barrier that shields the core pellet from environmental factors and minimizes incompatibility between the inert core and later applied drug or functional coatings. Commonly used seal coating 104 materials include polymers such as povidone K-30, hydroxypropyl cellulose, and hydroxypropyl methylcellulose (HPMC) at concentrations typically ranging from 3% to 5%. The seal coating 104 solution is usually water-based and requires careful control of process parameters like pellet bed temperature (around 40-45 °C) and inlet air temperature (45-55 °C) to ensure proper drying and uniform application.

In an embodiment, the drug-layered coating 106 is performed by spraying the drug-binder dispersion onto an inert core that includes one or more inert particles.

In an embodiment, the MCC sphere 108 has a particle size distribution characterized by a D90 value in the range of 350 to 500 micrometers (mm). The term ‘D90’ refers to the particle diameter below which 90% of the total volume of particles falls. MCC spheres 108, with a D90 of 350-500 mm, were selected as a suitable inert substrate for drug-layered coating 106.

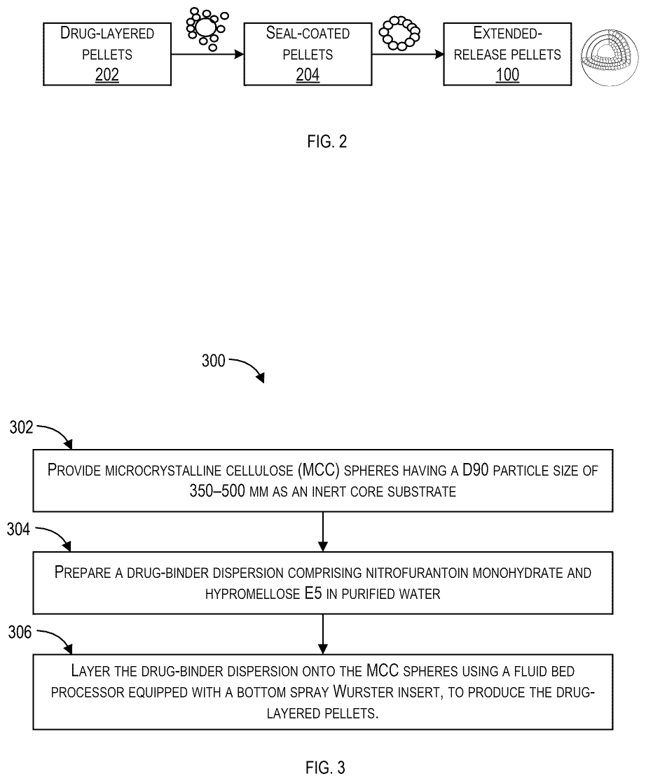

FIG. 2 is a flowchart of producing nitrofurantoin hybrid extended-release pellets 100 in accordance with an embodiment of the present disclosure. FIG. 2 shows the procedural steps for making nitrofurantoin hybrid extended-release pellets 100, including drug-layered pellets 202, and the drug-layered pellets 202 including nitrofurantoin API. In general, pharmaceutical pellets are small and spherical particles that are made by accumulating drug substances (mainly in the form of fine powders and granules) and excipients with the help of many processing and advanced technologies. The drug-layered pellet 202 formulation is a crucial process based on multiple unit-controlled release dosage forms to ensure uniform distribution of active drug substances in the human body. Factors such as composition, size, shape, coating, and excipients are considered in the formulation process. The composition controls efficiency, stability, patient acceptability, and dosage flexibility, while size affects compaction and drug release. Smaller pellets are preferred for quick release and absorption due to their large surface area to volume ratio. Coating is a crucial step in pellet formulation, applied to prolong the drug release and to increase the shelf life, mask taste and smell, and enhance stability. Excipients are essential for patient acceptability and shelf life.

In an embodiment, FIG. 2 shows the procedural steps for making nitrofurantoin hybrid extended-release pellets 100, including seal-coated pellets 204, which are prepared by spraying the seal coating dispersion onto drug-layered pellets 202 using Freund Vector VFC-LAB 1 fluid bed processors.

In an embodiment, the pharmaceutical composition includes an antibiotic formulated for extended-release by embedding an active ingredient in a matrix of soluble and/or insoluble substances. Soluble matrices, such as those composed of hydrophilic polymers including hydroxypropyl methylcellulose (HPMC), polyethylene oxide, or natural gums, may erode over time to release the drug. Insoluble matrix materials may include, but are not limited to, acrylic polymers, ethylcellulose, or chitin derivatives, which provide a diffusion-controlled release of the antibiotic. An extended-release form delivers the antibiotic at a predetermined rate, thereby maintaining effective therapeutic concentrations over an extended duration and potentially reducing dosing frequency.

In an embodiment, the term “Nitrofurantoin” includes nitrofurantoin drug and associated pharmaceutically acceptable salts and hydrates. “Macrocrystalline nitrofurantoin” refers to particulate crystalline nitrofurantoin, for example, as described in the United States Pharmacopoeia. Nitrofurantoin is a small molecule with a chemical name of 1-[(5-nitrofurfurylidene) amino] hydantoin, which is hydrophilic but has poor solubility in water. Used to treat urinary tract infections (UTIs) and targeted pathogens like E. coli, Enterococcus faecalis, Klebsiella spp., and Staphylococcus saprophyticus. In an embodiment, nitrofurantoin's mechanism of action involves reducing bacterial flavoproteins to reactive intermediates, which damage bacterial DNA, ribosomal proteins, and other macromolecules, resulting in bactericidal activity. Nitrofurantoin's mechanism is multi-targeted, reducing the risk of bacterial resistance. Nitrofurantoin is important for its effective treatment of lower UTIs and broad-spectrum activity against both gram-positive and gram-negative uropathogens.

In an embodiment, nitrofurantoin is used as an active pharmaceutical ingredient (API). The API is a primary component of a medicine that has the intended medical benefit. The API is the physiologically active ingredient that cures, diagnoses, or stops illness.

In an embodiment, nitrofurantoin drug release follows a sustained-release profile over 24 hours.

FIG. 3 illustrates a process 300 for creating drug-layered pellets 202, in accordance with an embodiment of the present disclosure. The process 300 for creating drug-layered pellets 202. At step 302, the process includes providing MCC spheres 108 having a D90 particle size of 350-500 mm as an inert core substrate.

In an embodiment, the drug-layered pellets 202 are prepared by drug-layered coating 106. The drug layering includes a microcrystalline cellulose (MCC) sphere 108 having a particle size distribution characterized by a D90 value in the range of 350 to 500 micrometers (mm). The term ‘D90’ refers to the particle diameter below which 90% of the total volume of particles falls. In an embodiment, the microcrystalline cellulose (MCC) spheres 108, with a D90 of 350-500 mm, were selected as a suitable inert substrate for drug-layered coating 106.

In an embodiment, at step 304, the process includes preparing a drug-binder dispersion comprising nitrofurantoin monohydrate and Methocel E5 Premium LV in purified water.

In an embodiment, nitrofurantoin monohydrate, which is a water-insoluble compound having pH-dependent solubility, is selected as the active pharmaceutical ingredient (API). The nitrofurantoin shows minimal solubility under acidic conditions and exhibits increased solubility at pH values above 6.8.

In an embodiment, the Methocel E5 Premium LV is selected as a suitable binder for the drug layering process due to compatibility with the drug and film-forming properties. Considering the solubility characteristics of the drug and the binder, purified water is selected as an appropriate solvent for preparing a homogenous drug-binder dispersion.

In an embodiment, a batch of MCC spheres 108 is taken as the initial substrate load for the drug-layered coating 106. Upon completion of the layering process, the batch size increased due to the deposition of the drug-binder composition. The process is designed to achieve a target drug layering corresponding to the total weight of the nitrofurantoin dispersion applied onto the MCC spheres 108.

In an embodiment, at step 306, the process includes layering the drug-binder dispersion onto the MCC spheres 108 using the Freund Vector VFC-LAB 1fluid bed processor equipped with a bottom spray Wurster insert, to produce the drug-layered pellets 202.

In an embodiment, the drug-layered coating 106 is carried out by spraying the prepared dispersion onto the MCC spheres 108. The spraying process is conducted using the Freund Vector VFC-LAB 1 fluid bed processor equipped with a Wurster coating assembly, providing uniform and precisely controlled drug layer deposition onto the substrate particles.

In an embodiment, the drug-layered coating 106 is carried out by the Freund Vector VFC-LAB 1fluid bed processor for coating pellets. In the coating process, the solution is sprayed into a bed, and pellets 100 are covered by the solution and dried by hot air. The coating layer on pellets can mask tastes, extend release, and enhance stability. The fluidised bed processor has a concentric cylindrical tube, which is called the Wurster insert, inside a conical chamber. The Wurster fluidised bed includes two flow regions: the coating region (inside the Wurster insert) and the annular region (outside the Wurster insert). In an embodiment, the flow is characterized by pneumatic mass transport circulation in the bed. During the circulation, the surface of pellets 100 is continuously wetted by the coating solution from a spray nozzle at the bottom and dried by hot air. At the end of the process, a coating layer is deposited onto the pellets 100. The advantage of fluidized bed coating is that it is suitable for uniformly coating pellets with different sizes and shapes, and both coating and drying processes are kept in one chamber.

In an embodiment, at the first step 302, provide microcrystalline cellulose (MCC) spheres 108 having a D90 particle size of 350-500 μm as an inert core substrate, then step 304 illustrates preparing a drug-binder dispersion comprising nitrofurantoin monohydrate and Methocel E5 premium LV in purified water and step 306 layer the drug-binder dispersion onto the MCC spheres 108 using the Freund Vector VFC-LAB 1fluid bed processor. The processor is equipped with a bottom spray Wurster insert to produce the drug-layered pellets.

In an embodiment, as exemplified by the process 300 for creating drug-layered pellets 202, a batch size of 1 kg of MCC spheres 108 is used as the initial substrate load for the drug. Upon completion of the layering process, the final batch size reached 2 kg, reflecting the addition of the drug-binder composition. The target drug loading for this process was set at 100% w/w with respect to the nitrofurantoin dispersion applied onto the MCC spheres 108.

In an embodiment, the drug layering composition, as taken exemplarily, includes the following:

| | | Quantity per batch |
|---|---|---|
| Material/Grade | Function | (g) |
| MCC spheres (Cellets ® 350) | Inert substrate | 1000.0 |
| Nitrofurantoin monohydrate | API | 950.0 |
| USP | | |
| Hypromellose USP | Binder | 50.0 |
| (Methocel E5 Premium LV) | | |
| Purified Water | Solvent | 2333.0 |

In an embodiment, an exemplary process 300 for creating drug-layered pellets 202, is prepared using materials per batch: approximately 1000.0 g of MCC spheres 108; approximately 950.0 g of nitrofurantoin monohydrate USP, functioning as the active pharmaceutical ingredient (API); 50.0 g Methocel E5 Premium LV, acting as a binder; and approximately 2333.0 g of purified water, serving as a solvent.

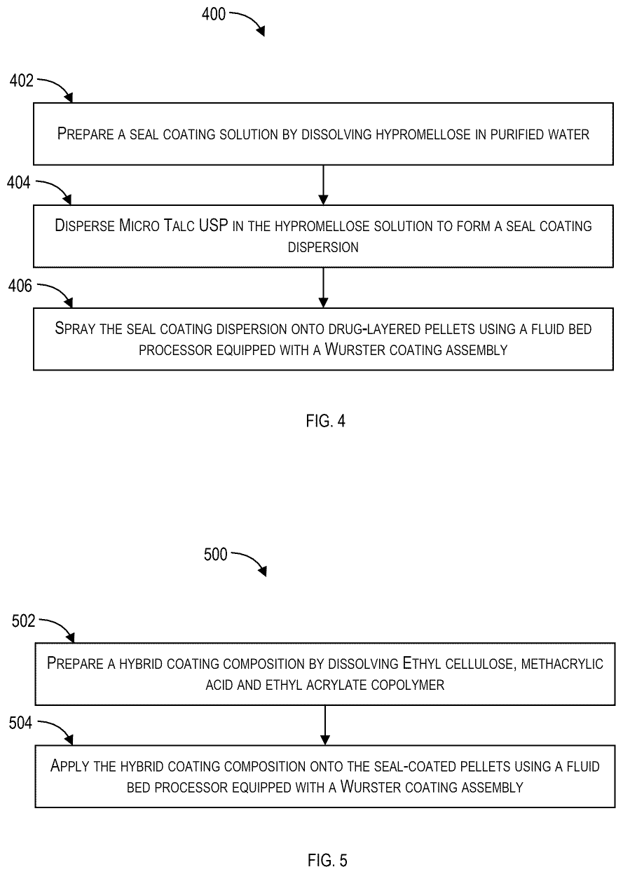

FIG. 4 illustrates a process 400 for creating seal-coated pellets 204, in accordance with an embodiment of the present disclosure. At step 402, the process includes preparing a seal coating solution by dissolving Methocel E5 Premium LV in purified water.

In an embodiment, a seal coating 104 is performed on the drug-layered pellets 202 before applying the hybrid extended-release coating 102 compositions. The seal coating 104 is applied to the drug-layered pellets 202 to provide a smooth and uniform surface for subsequent coating processes and to minimize any potential interaction between the active ingredient layer and the extended-release polymer coating to be applied further.

In an embodiment, Methocel E5 Premium LV is selected as a suitable seal coating 104 polymer. The methocel E5 premium LV is an inert, water-soluble polymer that forms a clear and uniform film upon drying, enhancing surface properties of the drug-layered pellets 202. Talc is selected as a suitable anti-tacking agent to prevent pellet agglomeration during the coating process and to ensure uniform coating distribution. Purified water is selected as the solvent for preparing the seal coating 104 dispersion based on its compatibility with both the drug and the selected excipients.

In an embodiment, at step 404, dispersion of micro talc USP in the methocel E5 premium LV solution to form a seal coating dispersion is done. Talc is gradually added to the methocel E5 premium LV solution and uniformly dispersed to form a stable aqueous dispersion suitable for coating application.

In an embodiment, at step 406, spraying the prepared hypromellose-talc dispersion onto the drug-layered pellets 202 using the Freund Vector VFC-LAB 1fluid bed process or equipped with a Wurster coating assembly. The Wurster configuration facilitated uniform coating of individual pellets by promoting consistent fluidization and efficient deposition of the coating dispersion.

In an embodiment, an exemplary seal coating process 400 is conducted to evaluate feasibility under targeted conditions. In this example, the starting batch load consisted of 1.000 kg of nitrofurantoin drug-layered pellets 202, resulting in a final batch size of 1.100 kg upon completion of the process. The target seal coating 104 level for this evaluation is 10% w/w. The composition of the seal coating dispersion included nitrofurantoin drug-layered pellets 202 (1000 g) as the core substrate, methocel E5 premium LV used as the seal coating polymer at 80 g per batch, and 20 g per batch micro talc USP used as an anti-tacking agent. The detailed composition used during this exemplary seal coating process 400 is presented in the tables below.

| | | Quantity per batch |
|---|---|---|
| Material/Grade | Function | (g) |
| Nitrofurantoin Drug- | Drug-loaded pellets | 1000 |
| layered pellets | | |
| Hypromellose USP | Seal coating polymer | 80 |
| Micro Talc USP | Anti-tacking agent | 20 |
| Purified Water | Solvent | 1900 |

FIG. 5 illustrates a process 500 for the hybrid extended-release coating 102, in accordance with an embodiment of the present disclosure.

In an embodiment, at step 502, prepare a hybrid coating composition by dissolving ethyl cellulose and methacrylic acid-ethyl acrylate copolymer.

In an embodiment, a hybrid coating composition is applied on previously seal-coated 204 nitrofurantoin drug-layered pellets 202 to achieve a dual-release profile incorporating both an extended-release (ER) and a delayed-release (DR) characteristic. The hybrid coating is applied as a single layer containing both an ER polymer and a DR polymer to optimize the desired drug release kinetics.

In an embodiment, the composition 502 of the hybrid coating includes two functional polymers: Ethyl cellulose, also known as Ethocel, which is selected as the extended-release polymer. Ethocel is a water-insoluble polymer known to form a semi-permeable matrix, thereby modulating drug diffusion and enabling extended drug release.

In an embodiment, a methacrylic acid-ethyl acrylate copolymer is used as the delayed or release polymer, also known as Eudragit L100-55. This polymer dissolves at pH>5.5, facilitating site-specific release in the intestinal environment. A single organic solvent, ethanol, is used to co-dissolve both polymers. To enhance the flexibility and mechanical integrity of the coating film, dibutyl sebacate is included as a suitable plasticizer, which also assists in preventing film cracking during curing and handling. In an embodiment, talc is incorporated as an anti-tacking agent to prevent agglomeration and ensure uniform coating during the process.

In an embodiment, 502, the hybrid coating composition is prepared by sequentially dissolving Ethocel and Eudragit L100-55 in ethanol under continuous stirring to ensure complete solubilization. Following polymer dissolution, dibutyl sebacate is added and thoroughly mixed. Talc is then slowly introduced into the solution while maintaining agitation to achieve uniform dispersion.

In an embodiment, at step 504, the hybrid coating composition is applied onto the seal-coated pellets 204 using the Freund Vector VFC-LAB 1fluid bed processor equipped with a Wurster coating assembly.

In an embodiment, the hybrid coating composition is applied on the seal-coated pellets 204 by using the Freund Vector VFC-LAB 1fluid bed processor equipped with a Wurster bottom-spray coating assembly. The seal-coated pellets 204 are first screened to remove fines and ensure a uniform starting substrate. The hybrid coating composition is sprayed onto the fluidized bed of pellets under controlled process parameters (e.g., atomizing air pressure, product temperature, inlet air temperature, spray rate, and fluidization airflow)

In an embodiment, the coating is applied until a total weight gain of up to 20% w/w is achieved, based on the initial weight of the seal-coated pellets 204. During the coating operation, intermediate samples were collected at coating levels of approximately 15% w/w and 18% w/w to monitor the progression of the hybrid extended-release profile and evaluate the impact of coating thickness.

In an embodiment, an exemplary hybrid extended-release (ER) coating dispersion is prepared and applied to nitrofurantoin seal-coated pellets 204 to achieve a hybrid ER and delayed-release (DR) profile. The coating formulation consisted of 1000.0 g of previously seal-coated pellets 204 as the substrate for providing extended-release, and 128.0 g of ethyl cellulose was used as the primary ER polymer. In addition, 32.0 g of methacrylic acid-ethyl acrylate copolymer is incorporated as the delayed-release polymer component to facilitate pH-dependent dissolution. The formulation further includes 20.0 g of dibutyl sebacate, functioning as a plasticizer to enhance film flexibility and mechanical stability. In an embodiment, to minimize tackiness during the coating process, 20.0 g of micro talc is used as an anti-tacking agent. Ethanol is utilized as the solvent in a quantity of 3800.0 g to dissolve both polymer components and ensure homogeneous dispersion of excipients.

In an embodiment, the extended-release polymer mixture comprises 80% ethyl cellulose and 20% methacrylic acid-ethyl acrylate copolymer. This ratio was optimized to balance the dual objectives of providing sustained release over a 24-hour period while minimizing premature drug release in acidic gastric conditions. A higher proportion of ethyl cellulose provides tighter diffusion control and extends the drug release curve, whereas the incorporation of 20% enteric copolymer enables a pH-responsive release onset, contributing to the desired site-specific intestinal delivery. Comparative trials with alternative ratios (e.g., 60:40 and 95:5) showed that 80:20 achieved the most consistent and reproducible extended-release profile, with acceptable coating process parameters and mechanical film integrity. In an embodiment, the exemplary parameters used during the application of the hybrid ER coating onto the seal-coated pellets 204 are provided in the table below.

| | | Quantity per batch |
|---|---|---|
| Material/Grade | Function | (g) |
| Nitrofurantoin seal-coated | Seal-coated pellets | 1000.0 |
| pellets | | |
| Ethocel Std 10 Premium | ER polymer | 128.0 |
| Methacrylic acid-ethyl | DR polymer | 32.0 |
| acrylate copolymer (Eudragit | | |
| L100-55) | | |
| Dibutyl Sebacate | Plasticizer | 20.0 |
| Micro Talc USP | Anti-tacking agent | 20.0 |
| Ethanol 190 pf | Solvent | 3800.0 |

In an embodiment, the ethyl cellulose acts as a release-controlling polymer, and a combination of methacrylic acid-ethyl acrylate copolymer acts as a delayed release polymer or enteric coating polymer. The release-controlling polymer comprises 60%-95% of the mixture, and the delayed release polymer comprises 5%-40% of the mixture.

In an embodiment, ethyl cellulose polymer comprises 80% by weight of the total polymer mixture, and methacrylic acid-ethyl acrylate copolymer comprises 20% by weight of the total polymer mixture.

In an embodiment, the composition of coated layer initially comprises a solution containing 128.0 mg of ethyl cellulose as an extended-release polymer, 32.0 mg of methacrylic acid-ethyl acrylate copolymer (1:1) as a delayed-release polymer, 20.0 mg of dibutyl sebacate as a plasticizer, 20.0 mg of Micro Talc USP as an anti-tacking agent, and 3800.0 mg of ethanol as a volatile solvent. Ethanol serves as a processing solvent and is evaporated during the coating process. Accordingly, ethanol does not contribute to the final weight of the coated layer on the dosage form.

In an embodiment, upon drying, the hybrid extended-release coating comprises a total of 200.0 mg of non-volatile solid components. In the final dry coating layer, the relative composition by weight consists of approximately: the release-controlling polymer is 64.0% of the hybrid extended-release coating 102 composition, the delayed-release polymer is 16.0% of the hybrid extended-release coating 102 composition, dibutyl sebacate is 10.0% of the hybrid extended-release coating 102 composition, and micro talc USP is 10.0% of the hybrid extended-release coating 102 composition.

In an embodiment, the hybrid extended-release coating 102 is applied to achieve a weight gain of up to 20% w/w.

In an embodiment, optionally, one or more additional water-soluble polymers may be incorporated into the coating system to further modulate the drug release profile. These polymers can serve multiple functional roles, such as enhancing film permeability, adjusting coating porosity, or facilitating controlled hydration and erosion of the coating matrix upon contact with gastrointestinal fluids. The inclusion of water-soluble polymers may allow for fine-tuning of the lag time, onset of release, and overall release kinetics, particularly in formulations designed for modified, sustained, or pulsatile drug delivery. The type, molecular weight, and concentration of the selected polymer(s) may be varied depending on the desired release characteristics and compatibility with other coating components.

In an embodiment, besides the above polymers, the sustained release portion may also contain other pharmaceutically acceptable excipients such as substrate/inert core/nonpareil beads, binder/film forming agent, anti-tacking agent, surfactant, water insoluble drug release controlling polymer, delayed release enteric polymer, plasticizer, pore-former.

In an embodiment, a suitable substrate/inert core may include MCC spheres, sugar spheres, tartaric acid pellets, calcium phosphate pellets, and an isomalt-type pellet core. The suitable binders may include Hypromellose, acacia, agar, alginic acid, carbomer, chitosan, polyvinyl alcohol, copovidone, gelatin, hydroxyethyl cellulose, hydroxypropyl cellulose, maltodextrin, polydextrose, polyethylene glycol, polyethylene oxide, polymethacrylates, poly (methylvinyl ether/maleic anhydride), polyvinyl acetate phthalate, shellac, starch. The suitable anti-tacking agent may include Talc, colloidal/fumed silicon dioxide, magnesium stearate, stearic acid, sodium stearyl fumarate, magnesium oxide, magnesium silicate, magnesium trisilicate, glyceryl monostearate.

In an embodiment, a suitable surfactant may include polysorbate, sodium lauryl sulfate, cetyltrimethylammonium bromide, carbomer, benzalkonium chloride, or poloxamer. The suitable water-insoluble drug release controlling polymer may include ethyl cellulose, cellulose acetate, cellulose acetate butyrate, cellulose diacetate, cellulose triacetate, cellulose propionate, wax, copolymer of ethyl arylate-methyl methacrylate-trimethylammonioethyl methacrylate chloride (1:2:0.1), copolymer of ethyl arylate-methyl methacrylate-trimethylammonioethyl methacrylate chloride (1:2:0.2), copolymer of ethyl acrylate-methyl methacrylate, polyvinyl acetate dispersion, shellac, alginates, cellulose acetate succinate, cellulose acetate phthalate, methacrylic acid-ethylacrylate copolymer (1:1).

In an embodiment, the delayed release enteric polymer may include cellulose acetate phthalate, poly (methacrylic acid-comethyl methacrylate), cellulose acetate trimellitate, poly(vinyl acetate phthalate), hydroxypropyl methylcellulose phthalate, and shellac (esters of aluretic acid). The suitable plasticizer may include dibutyl sebacate, dimethyl phthalate, diethyl phthalate, dibutyl phthalate, hypromellose, glycerin, polyethylene glycol monomethyl ether, triethyl citrate, acetyl triethyl citrate, tributyl citrate, acetyl tributyl citrate, castor oil, diacetylated monoglycerides, triacetin, polyethylene glycols, propylene glycol, sorbitol sorbitan solution, polyethylene glycol monomethyl ether, glycerin.

In an embodiment, as an exemplary dissolution test of nitrofurantoin multiparticulates is done on drug layered pellets 202 is performed in USP apparatus II, with an agitation speed of 50 rpm, and the dissolution medium is 900 mL of 0.1N HCl. Dissolution is performed for up to 90 minutes, and samples were quantified using a UV spectrophotometer at 375 nm wavelength.

In an embodiment, the dissolution data is shown in the table below:

| | Immediate release Drug |
|---|---|
| | layered pellets |
| Time in mins | % Drug released |
| 0 | 0 |
| 20 | 27 |
| 30 | 33 |
| 45 | 37 |
| 60 | 42 |
| 90 | 45 |

In an embodiment, as an example, the dissolution testing is performed for the nitrofurantoin hybrid coated ER pellets 100 using the USP Apparatus II, with 900 mL 0.1 N HCl as dissolution media for 2 hours, followed by pH 7.5 media for 24 hours. The pellets equivalent to 200 mg nitrofurantoin are weighed and loaded into the dissolution vessel. The paddle speed used is 50 RPM. The dissolution sampling time intervals include 0.5, 1, 2, 4, 8, 12, 16, 18, and 24 hours. Dissolution samples are tested using a UV spectrophotometer at 375 nm wavelength.

In an embodiment, nitrofurantoin hybrid ER coated pellets 100 with the coating level/weight gain of 15%, 18%, and 20% are evaluated for dissolution at the dose level of 200 mg.

In an embodiment, the dissolution data for the hybrid ER-coated pellets 100 is shown in the table below:

| | Nitrofurantoin ER | Nitrofurantoin ER | Nitrofurantoin ER |
|---|---|---|---|
| | Pellets, 15% ER | Pellets, 18% ER | Pellets, 20% ER |
| Time | coating weight gain | coating weight gain | coating weight gain |
| in hrs. | % Drug released | % Drug released | % Drug released |
| 0 | 0 | 0 | 0 |
| 0.5 | 1 | 0 | 1 |
| 1 | 2 | 0 | 0 |
| 2 | 4 | 1 | 2 |
| 4 | 23 | 22 | 18 |
| 8 | 44 | 40 | 35 |
| 12 | 57 | 54 | 47 |
| 16 | 76 | 67 | 60 |
| 18 | 90 | 82 | 75 |
| 24 | 97 | 90 | 86 |

In an embodiment, the dissolution profile slows down with the increase in the weight gain of the ER-coated pellets 100, confirming that the coating is uniform and drug release can be tailored based on the ER coating 102 weight gain.

In an embodiment, as an exemplary test done data and dissolution results, 200 mg per unit dose is successfully demonstrated for negligible drug release in acid media and a gradual increase in buffer media over the period of time. The in vitro drug release of up to 24 hours is promising and can potentially provide a once-a-day dosing regimen for the nitrofurantoin ER capsules, while eliminating premature drug release in acid media.

In an embodiment, once daily ER capsule will deliver nitrofurantoin at a predetermined rate for a pre-specified period, reducing peak-to-trough fluctuations while maintaining therapeutically effective nitrofurantoin plasma and urine concentrations, therefore providing better coverage throughout the day, maximizing the clinical effectiveness.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprise” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . , and N, the text should be interpreted as requiring only one element from the group, not A plus N, or B plus N, etc.

While the foregoing describes various embodiments of the invention, other and further embodiments of the invention may be devised without departing from the basic scope thereof. The scope of the invention is determined by the claims that follow. The invention is not limited to the described embodiments, versions, or examples, which are included to enable a person having ordinary skill in the art to make and use the invention when combined with information and knowledge available to the person having ordinary skill in the art.

What is claimed is:

1. A pharmaceutical composition comprising:
drug-layered pellets containing nitrofurantoin as an active pharmaceutical ingredient (API); and
a hybrid extended-release coating composition surrounding each of the drug-layered pellets, the extended-release coating composition comprises a polymer mixture of 60%-95% release-controlling polymer and 05%-40% delayed release polymer, wherein the release-controlling polymer is ethyl cellulose and the delayed release polymer is a combination of methacrylic acid-ethyl acrylate copolymer.

2. The pharmaceutical composition of claim 1, wherein the extended-release coating composition further comprises 1-30% dibutyl sebacate serving as a plasticizer, micro talc USP serving as an anti-tacking agent, and ethanol serving as a solvent.

3. The pharmaceutical composition of claim 1, wherein the release-controlling polymer comprises 80% by weight of the total polymer mixture, and the delayed release polymer comprises 20% by weight of the total polymer mixture.

4. The pharmaceutical composition of claim 1, wherein the release-controlling polymer is 64.0% of the extended-release coating composition, the delayed release polymer is 16.0% of the extended-release coating composition, dibutyl sebacate is 10.0% of the extended-release coating composition, micro talc USP is 10.0% of the extended-release coating composition, calculated on a dry weight basis after solvent ethanol is evaporated.

5. The pharmaceutical composition of claim 1, wherein the extended-release coating is applied to achieve a weight gain of up to 20% w/w.

6. The pharmaceutical composition of claim 1, wherein each of the drug-layered pellets is prepared using a Freund Vector VFC-LAB 1 fluid bed processor equipped with a Wurster coating assembly.

7. The pharmaceutical composition of claim 1, wherein the nitrofurantoin release follows a sustained-release profile over 24 hours.

8. The pharmaceutical composition of claim 1, wherein the drug-layered pellets are prepared by:
providing microcrystalline cellulose (MCC) spheres having a D90 particle size of 350-500 mm as an inert core substrate;
preparing a drug-binder dispersion comprising nitrofurantoin monohydrate and Methocel E5 Premium LV also known as Hydroxypropyl methylcellulose (HPMC), in purified water; and
layering the drug-binder dispersion onto the MCC spheres using the Freund Vector VFC-LAB 1 fluid bed processor equipped with a bottom spray Wurster insert, to produce the drug-layered pellets.

9. The pharmaceutical composition of claim 1, wherein seal coating is performed on the drug-layered pellets before applying the extended-release coating composition.

10. The pharmaceutical composition of claim 1, wherein the extended-release coating composition is sprayed on the drug-layered pellets.

11. A method for preparing extended-release nitrofurantoin pellets, the method comprising:
preparing drug-layered pellets comprising nitrofurantoin as an active pharmaceutical ingredient (API);
applying a seal coating over the drug-layered pellets to obtain sealed-coated pellets; and
applying extended-release coating composition over the seal-coated pellets, wherein the extended-release coating composition comprises a polymer mixture of 60%-95% release-controlling polymer and 5%-40% delayed release polymer, wherein the release-controlling polymer is ethyl cellulose and the delayed release polymer is a combination of methacrylic acid-ethyl acrylate copolymer.

12. The method of claim 11, wherein the extended-release coating composition further comprises 1-30% dibutyl sebacate serving as a plasticizer, micro talc USP serving as an anti-tacking agent, and ethanol serving as a solvent.

13. The method of claim 11, wherein the release-controlling polymer is 64.0% of the extended-release coating composition, the delayed release polymer is 16.0% of the extended-release coating composition, dibutyl sebacate is 10.0% of the extended-release coating composition, micro talc USP is 10.0% of the extended-release coating composition, calculated on a dry weight basis after solvent ethanol is evaporated.

14. The method of claim 11, wherein the drug-layered pellets are prepared by:
providing microcrystalline cellulose (MCC) spheres having a D90 particle size of 350-500 mm as an inert core substrate;
preparing a drug-binder dispersion comprising nitrofurantoin monohydrate and Methocel E5 Premium LV in purified water; and
layering the drug-binder dispersion onto the MCC spheres, using the Freund Vector VFC-LAB 1fluid bed processor equipped with a bottom spray Wurster insert, to produce the drug-layered pellets.

15. The method of claim 11, wherein the seal coating is applied on drug-layered pellets by:
preparing a dispersion solution by dissolving Methocel E5 Premium LV in purified water;
adding micro talc USP in the dispersion solution to form a seal coating dispersion, wherein the methocel E5 premium LV is an inert water-soluble polymer and the microtalc USP is an anti-tacking agent; and
spraying the seal coating dispersion onto the drug-layered pellets, the said seal coating is applied to achieve a target coating level of approximately 10% w/w.

16. The method of claim 11, wherein the extended-release coating is applied by:
preparing a mixture by dissolving ethyl cellulose and methacrylic acid-ethyl acrylate copolymer into ethanol;
adding dibutyl sebacate, and talc in the mixture, wherein the dibutyl sebacate is added to the mixture acting as a plasticizer; and the talc is used as an anti-tacking agent to form the extended-release coating composition; and
applying the hybrid coating composition onto the seal-coated pellets using the Freund Vector VFC-LAB 1fluid bed processor equipped with a Wurster coating assembly, wherein the coating process is continued until a weight gain of up to 20% w/w is achieved.

17. The method of claim 11, wherein the release-controlling polymer comprises 80% by weight of the total polymer mixture; and the delayed release polymer comprises 20% by weight of the total polymer mixture.

18. The method of claim 11, wherein the extended-release nitrofurantoin pellets release follows a sustained-release profile over 24 hours once administered.
